(19) United States
(12) Patent Application Publication
(10) Pub. No.: US 2022/0088576 A1
(43) Pub. Date: Mar. 24, 2022

(54) Self-Disinfecting Multi-Band Photocatalyst Sheet

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/103,461

(22) Filed: Nov. 24, 2020

Related U.S. Application Data
(63) Continuation-in-part of application No. 17/027,535, filed on Sep. 21, 2020.

(51) Int. Cl. B01J 23/50; B01J 21/06; B01J 23/10; B01J 35/00; C09J 7/29; C09J 11/04; A61L 2/08; A61L 2/10

(52) U.S. Cl. CPC B01J 23/50; B01J 21/063; B01J 23/10; B01J 35/004; C09J 7/29; C09J 11/04; C09J 2427/006; A61L 2/10; A61L 2/084; C09J 2301/302; C09J 2301/41; C09J 2400/143; A61L 2/088

(57) ABSTRACT

A self-disinfecting photocatalyst sheet includes a substrate material and a photocatalyst layer with a primary photocatalyst and a secondary photocatalyst. The primary photocatalyst is a metal oxide photocatalyst, whereas the secondary photocatalyst is a metallic photocatalyst. The primary photocatalyst forms a covalent bond with the substrate material. The self-disinfecting photocatalyst sheet is photocatalytic active to different bands of wavelength. Another self-disinfecting photocatalyst sheet includes a substrate material, a prime material layer and a photocatalyst layer with a primary photocatalyst and a secondary photocatalyst. The prime material layer is between the substrate and the photocatalyst layer. The primary photocatalyst forms a covalent bond with the prime material.

### CROSS-REFERENCE TO RELATED PATENT APPLICATION(S)

The present disclosure is a continuation-in-part (CIP) of U.S. patent application Ser. No. 17/027,535, filed 21 Sep. 2020, the content of which being incorporated by reference in its entirety herein.

### BACKGROUND

### Technical Field

The present disclosure pertains to the field of antimicrobial photocatalyst device and, more specifically, proposes a self-disinfecting multi-band (multi-wavelength range) photocatalyst sheet.

### Description of Related Art

In U.S. patent application Ser. No. 17/027,535, a self-disinfecting photocatalyst sheet was introduced. It includes a substrate material and a photocatalyst layer with a primary photocatalyst and a secondary photocatalyst. The primary photocatalyst is a metal oxide photocatalyst, whereas the secondary photocatalyst is a metallic photocatalyst. The primary photocatalyst forms a covalent bond with the substrate material. The self-disinfecting photocatalyst sheet is activatable by a visible light and can self-disinfect against bacteria and viruses. One area that was not properly addressed by U.S. patent application Ser. No. 17/027,535 is the photocatalytic nature of the photocatalyst layer in response to different bands of wavelength, in addition to the visible wavelength band in 400˜700 nm. Moreover, the photocatalytic activity level to different bands of wavelength may also vary from one wavelength band to another. This has importantly implication to the application of the self-disinfection photocatalyst sheet.

Another area that was not properly addressed by U.S. patent application Ser. No. 17/027,535 is the use of a prime material layer between the photocatalyst layer and the substrate material. A prime material may provide a stronger adhesion with the substrate material and at the same time form a solid covalent bond with the photocatalyst layer. These features would be beneficial when making the self-disinfection photocatalyst sheet on hard-to-adhere substrate materials.

### SUMMARY

In one aspect, the self-disinfecting photocatalyst sheet comprises a substrate material and a photocatalyst layer. The substrate material has a first side and a second side. The photocatalyst layer contains a primary photocatalyst and a secondary photocatalyst. The primary photocatalyst is a metal oxide photocatalyst, whereas the secondary photocatalyst is a metallic photocatalyst. The mass ratio of the primary photocatalyst to the secondary photocatalyst is greater than 2:1. The primary photocatalyst forms a covalent bond with the substrate material at the molecular level on the first side of the substrate material. The secondary photocatalyst which is a metallic photocatalyst may not form a valent boding with the substrate material. The photocatalyst layer is photocatalytic active to every wavelength band of a plurality of wavelength bands comprising 190˜280 nm, 280˜315 nm, 315˜400 nm, and 400˜700 nm. Moreover, the photocatalyst layer is more photocatalytic active to the wavelength band 190˜280 nm than to the wavelength band 280˜315 nm, when the photocatalyst layer is exposed to the same amount of germicidal light dosage (mJ/cm²) from both wavelength bands. Similarly, the photocatalyst layer is more photocatalytic active to the wavelength band 280˜315 nm than to the wavelength band 315˜400 nm. The photocatalyst layer is more photocatalytic active to the wavelength band 315˜400 nm than to the wavelength band 400˜700 nm.

The photocatalyst layer of the present disclosure is photocatalytic active to all wavelengths, and can thus be used in all lighting conditions, and does not require a dedicated light source with a specific wavelength band. However, in some application where a higher photocatalytic activity is desirable, an ultraviolet C (UVC) light source may be used, over an ultraviolet B (UVB) light, over an ultraviolet A (UVA) light source, over a visible light source. It is known that UVC can cause skin and eye damages to an occupant. American Conference of Governmental Industrial Hygienists (ACGIH) has published a UV Safety Guidelines as shown in FIG. 1 (ACGIH ISBN: 0-9367-12-99-6). It shows the UV Threshold Limit Values (TLVs), which is the maximum allowable dosage (in mJ/cm²) for each wavelength over an 8-hour period. It can be seen from FIG. 1, the TLV for 222 nm wavelength is set to 22mJ/cm², and the TLV for 254 nm wavelength is 6mJ/cm². Therefore, it is not recommended to administrate more 6mJ/cm² of 254 nm UVC wavelength for an 8-hour workday. It can also be seen from FIG. 1 that the TLV for UVB and UVA wavelength bands are much higher.

The present disclosure enables a user to trade between the photocatalytic activity and the UV dosage in the selection of a light source, and to find the best balance between them for any application. For example, the present disclosure can be used on a car window without any dedicated light source. This is because the sunlight has a full spectrum and can activate the photocatalyst layer perfectly. When the present disclosure is attached to a surface in an indoor environment, there is not enough sunlight exposure. Nonetheless, an electric light source in the indoor environment most likely emits 400 nm to 700 nm wavelength, and thus can activate the photocatalyst layer of the present disclosure to achieve a lower, maintenance-level self-disinfection for the surface of the present disclosure. At night, when there is no occupant in that indoor environment, it is possible to use a UVA or UVC light source to accelerate the photocatalytic activity of the photocatalyst layer of the present disclosure, thus achieving a thorough self-disinfection for the surface of the present disclosure. In this scenario, since the UVA or the UVC light source operates at night, there is no issue with UV over-dosage.

In some embodiments, the primary metal oxide photocatalyst includes anatase titanium dioxide (TiO₂) because the anatase-type titanium (IV) dioxide (TiO₂) is known to have the best photocatalytic property among TiO₂ material family.

In some embodiments, the secondary metallic photocatalyst may include silver, gold, copper, zinc, nickel, cerium, or a combination thereof. It is foreseeable to use other metal elements as the secondary photocatalyst. The metallic photocatalyst not only contributes to the photocatalytic activity itself, but also enables the primary metal oxide photocatalyst to absorb spectral energy in the visible light wavelength and become photocatalytic active.

The secondary metallic photocatalyst may contain one or two or even more metallic photocatalyst materials. In some embodiments, the secondary photocatalyst comprises two metallic photocatalyst materials, a third metallic photocatalyst and a fourth metallic photocatalyst. More specifically, in some embodiments, the third metallic photocatalyst comprises silver nanoparticles (NPs) and the fourth metallic photocatalyst comprises cerium NPs. Both silver NPs and cerium NPs help improve the photocatalytic activity of anatase TiO₂ when illuminated with a visible light. It is found that silver NPs themselves are effective in inhibiting bacteria under a visible light, whereas cerium NPs are effective in inhibiting viruses under a visible light. Having both silver NPs and cerium NPs as the secondary metallic photocatalyst would improve the self-disinfection effectiveness of the present disclosure under a visible light.

In some embodiments, the substrate material comprises a glass. Some of the cellphone screen protectors are made of soda lime glass or alkaline-aluminosilicate glass, and they can be made to be a very thin sheet. They would be good candidates for the substrate material of the present disclosure. A screen protector with a self-disinfecting photocatalytic surface provides the user of the cellphone a continual antimicrobial protection against any germs on the screen protector.

In some embodiments, the substrate material comprises a resin. The resin has been widely used for screen protector, packaging, and surface covering. Some widely used resin includes polyvinyl chloride, polyethylene, polyethylene terephthalate, polyurethane, thermoplastic polyurethane, polypropylene, polystyrene, silicone, and other thermoplastic and thermosetting resins. Once a resin forms a covalent band with the proposed primary photocatalyst, the surface of the resin product would become antimicrobial and self-disinfecting. Such resin could then be used for covering any surface, flat or not, and providing a continual disinfection protection for the surface.

In some embodiments, an adhesive layer is coated on the second side of the substrate material. In some embodiments, the optional adhesive layer comprises a pressure-sensitive adhesive (PSA) material. PSAs, especially acrylic based PSAs, may be tailored to provide a number of desirable attributes such as elasticity, tackiness, transparency, resistance to oxidation and sunlight, as well as have the necessary degree of adhesion and cohesion for different applications. With a PSA layer, the present disclosure may be attached, removed, and reattached to a surface, and provide a self-disinfection protection for the surface.

In some other embodiments, the optional adhesive layer comprises an electrostatic-enhancing agent for the purpose of adhering to a surface made of a material capable of being adhered to via electrostatic. Plastic, ceramic, and glass are materials that can be adhered to via electrostatic. The electrostatic-enhancing agent may be epoxy resin, polyepoxide, or graphene. It is foreseeable that the substrate material may contain an electrostatic-enhancing agent such as graphene, and in such case, it would not be necessary to add an electrostatic-enhancing agent layer to the substrate material.

In some embodiments, the present disclosure is transparent and has a transmission rate of visible light greater than 50%. It is foreseeable that additional material(s) may be added to the substrate material or additional coating(s) may be added on the second side of the substrate material for different applications, such as filtering out UV light or blue light, or adding tint to reduce the spectral penetration rate of a visible light.

In another aspect, the self-disinfecting photocatalyst sheet comprises a substrate material, a prime material layer, and a photocatalyst layer. The substrate material has a first side and a second side. The photocatalyst layer contains a primary photocatalyst and a secondary photocatalyst. The primary photocatalyst is a metal oxide photocatalyst, whereas the secondary photocatalyst is a metallic photocatalyst. The mass ratio of the primary photocatalyst to the secondary photocatalyst is greater than 2:1. The prime material layer is disposed between the substrate material and the photocatalyst layer. The primary photocatalyst forms a covalent bond with the prime material at a molecular level. The prime material layer is coated on the first side of the substrate material. The prime material helps providing a stronger adhesion with the substrate material and at the same time forming a solid covalent bond with the photocatalyst layer. The photocatalyst layer is photocatalytic active to every wavelength band of a plurality of wavelength bands comprising 190˜280 nm, 280˜315 nm, 315˜400 nm, and 400˜700 nm. Moreover, the photocatalyst layer is more photocatalytic active to the wavelength band 190˜280 nm than to the wavelength band 280˜315 nm, when the photocatalyst layer is exposed to the same amount of germicidal light dosage (mJ/cm²) from both wavelength bands. Similarly, the photocatalyst layer is more photocatalytic active to the wavelength band 280˜315 nm than to the wavelength band 315˜400 nm. The photocatalyst layer is more photocatalytic active to the wavelength band 315˜400 nm than to the wavelength band 400˜700 nm.

In some embodiments, the primary metal oxide photocatalyst includes anatase titanium dioxide (TiO₂) because the anatase-type titanium (IV) dioxide (TiO₂) is known to have the best photocatalytic property among TiO₂ material family.

In some embodiments, the secondary metallic photocatalyst may include silver, gold, copper, zinc, nickel, cerium, or a combination thereof. It is foreseeable to use other metal elements as the secondary photocatalyst. The metallic photocatalyst not only contributes to the photocatalytic activity itself, but also enables the primary metal oxide photocatalyst to absorb spectral energy in the visible light wavelength and become photocatalytic active.

The secondary metallic photocatalyst may contain one or two or even more metallic photocatalyst materials. In some embodiments, the secondary photocatalyst comprises two metallic photocatalyst materials, a third metallic photocatalyst and a fourth metallic photocatalyst. More specifically, in some embodiments, the third metallic photocatalyst comprises silver nanoparticles (NPs) and the fourth metallic photocatalyst comprises cerium NPs.

In some embodiments, the substrate material comprises a glass. In some other embodiments, the substrate material comprises a resin.

In some embodiments, an adhesive layer is coated on the second side of the substrate material. In some embodiments, the optional adhesive layer comprises a pressure-sensitive adhesive (PSA) material. In some other embodiments, the optional adhesive layer comprises an electrostatic-enhancing agent for the purpose of adhering to a surface made of a material capable of being adhered to via electrostatic.

In some embodiments, the present disclosure is transparent and has a transmission rate of visible light greater than 50%.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines

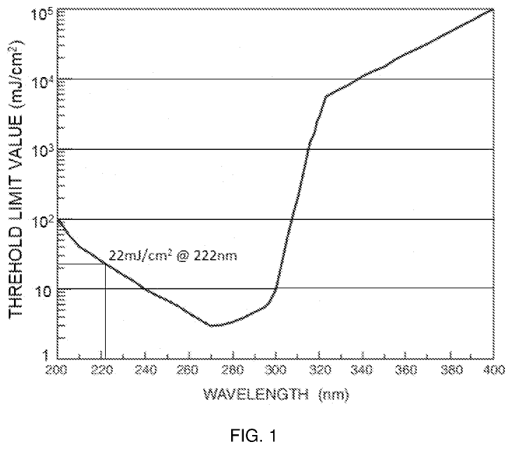

FIG. 2 schematically depicts a diagram of an embodiment of the present disclosure with adhesive coating on the second side of a substrate material.

FIG. 3 schematically depicts a diagram of another embodiment of the present disclosure with a prime material layer between the substrate and the photocatalyst layer.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of the primary, the secondary photocatalyst, the substrate material, and the prime material.

The present disclosure discloses self-disinfecting photocatalyst sheet includes a substrate material and a photocatalyst layer with a primary photocatalyst and a secondary photocatalyst. The primary photocatalyst is a metal oxide photocatalyst, whereas the secondary photocatalyst is a metallic photocatalyst. The primary photocatalyst forms a covalent bond with the substrate material. The self-disinfecting photocatalyst sheet is photocatalytic active to different bands of wavelength. Another self-disinfecting photocatalyst sheet includes a substrate material, a prime material layer and a photocatalyst layer with a primary photocatalyst and a secondary photocatalyst. The prime material layer is between the substrate and the photocatalyst layer. The primary photocatalyst forms a covalent bond with the prime material.

### EXAMPLE IMPLEMENTATIONS

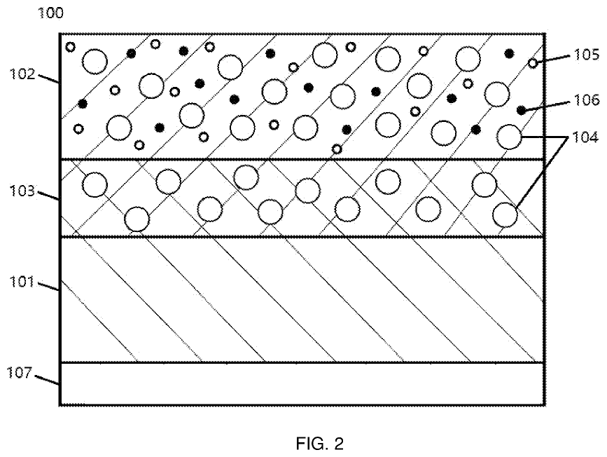

In FIG. 2, an embodiment 100 of the present disclosure is shown. A photocatalyst layer 102 is coated over a substrate material, polyvinyl chloride (PVC) 101. The photocatalyst layer contains a primary metal oxide photocatalyst TiO₂ 104 and two secondary metallic photocatalysts, silver nanoparticles (NPs) 105 and cerium NPs 106. The mass ratio of the primary photocatalyst TiO₂ 104 to the secondary metallic photocatalysts, silver nanoparticles (NPs) 105 and cerium NPs 106, is greater than 2:1. The primary photocatalyst TiO₂ 104 forms a covalent bond with the PVC substrate material 101 along the boundary 103 where the photocatalyst layer 102 meets the substrate material 101. With the presence of the two secondary photocatalysts, the photocatalyst layer 102 is photocatalytic active to all wavelength bands in 190˜280 nm, 280˜315 nm, 315˜400 nm, and 400˜700 nm, individually and concurrently. Moreover, the photocatalytic activeness of the photocatalyst layer 102 decreases from 190˜280 nm wavelength band to 280˜315 nm band, to 315˜400 nm band, to 400˜700 nm band. The second side of the PVC substrate material is coated with an adhesive layer 107 comprising a pressure-sensitive adhesive (PSA) material. With a PSA layer, the embodiment could be used as self-disinfecting window film, and it can be attached, removed, and even reattached to a glass window and provide self-disinfection protection for the glass window.

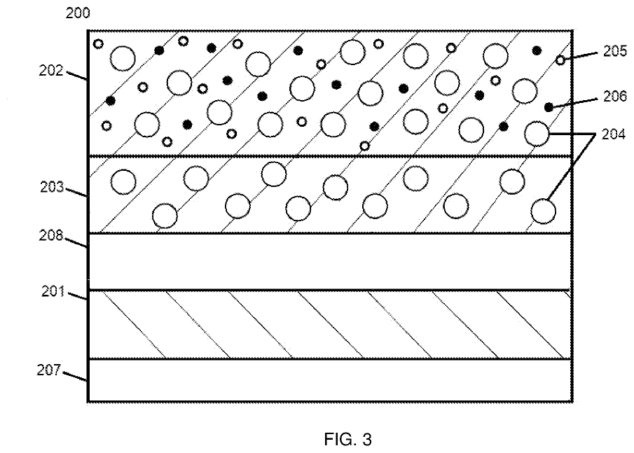

In FIG. 3, another embodiment 200 of the present disclosure is shown. A photocatalyst layer 202 is coated over a substrate material, polyvinyl chloride (PVC) 201. The photocatalyst layer contains a primary metal oxide photocatalyst TiO₂ 204 and two secondary metallic photocatalysts, silver nanoparticles (NPs) 205 and cerium NPs 206. The mass ratio of the primary photocatalyst TiO₂ 204 to the secondary metallic photocatalysts, silver nanoparticles (NPs) 205 and cerium NPs 206, is greater than 2:1. There is a prime material layer 208 between the substrate material 201 and the photocatalyst layer 202. The primary photocatalyst TiO₂ 204 forms a covalent bond with the prime material layer 208 along the boundary 203 where the photocatalyst layer 202 meets the prime material layer 208. With the presence of the two secondary photocatalysts, the photocatalyst layer 202 is photocatalytic active to all wavelength bands in 190˜280 nm, 280˜315 nm, 315˜400 nm, and 400˜700 nm, individually and concurrently. Moreover, the photocatalytic activeness of the photocatalyst layer 102 decreases from 190˜280 nm wavelength band to 280˜315 nm band, to 315˜400 nm band, to 400˜700 nm band. The second side of the PVC substrate material is coated with an adhesive layer 207 comprising a pressure-sensitive adhesive (PSA) material. In some implementation, the photocatalyst layer is more photocatalytic active to the wavelength band 190˜280 nm than to the wavelength band 280˜315 nm, when the photocatalyst layer is exposed to the same amount of germicidal light dosage (mJ/cm²) from both wavelength bands. Additionally, or alternatively, the photocatalyst layer is more photocatalytic active to the wavelength band 280˜315 nm than to the wavelength band 315˜400 nm, when the photocatalyst layer is exposed to the same amount of germicidal light dosage (mJ/cm²) from both wavelength bands. Additionally, or alternatively, the photocatalyst layer is more photocatalytic active to the wavelength band 315˜400 nm than to the wavelength band 400˜700 nm, when the photocatalyst layer is exposed to the same amount of germicidal light dosage (mJ/cm²) from both wavelength bands.

### ADDITIONAL AND ALTERNATIVE IMPLEMENTATION NOTES

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A self-disinfecting photocatalyst sheet, comprising
a substrate material with a first side and a second side opposite the first side; and
a photocatalyst layer comprising a primary photocatalyst and a secondary photocatalyst,
wherein:
the primary photocatalyst comprises a metal oxide photocatalyst,
the secondary photocatalyst comprises a metallic photocatalyst,
a mass ratio of the primary photocatalyst to the secondary photocatalyst is greater than 2:1,
the primary photocatalyst forms a covalent bond with the substrate material at a molecular level on the first side of the substrate material,
the photocatalyst layer is photocatalytic active to every wavelength band of a plurality of wavelength bands comprising 190˜280 nm, 280˜315 nm, 315 400 nm, and 400˜700 nm,
the photocatalyst layer is more photocatalytic active to the wavelength band 190˜280 nm than to the wavelength band 280˜315 nm,
the photocatalyst layer is more photocatalytic active to the wavelength band 280˜315 nm than to the wavelength band 315˜400 nm, and
the photocatalyst layer is more photocatalytic active to the wavelength band 315˜400 nm than to the wavelength band 400˜700 nm.

2. The self-disinfecting photocatalyst sheet of claim 1, wherein the primary photocatalyst further comprises anatase titanium dioxide (TiO₂).

3. The self-disinfecting photocatalyst sheet of claim 1, wherein the secondary photocatalyst further comprises silver, gold, copper, zinc, nickel, cerium, or a combination thereof.

4. The self-disinfecting photocatalyst sheet of claim 3, wherein the secondary photocatalyst further comprises a third metallic photocatalyst and a fourth metallic photocatalyst with no other metallic photocatalysts.

5. The self-disinfecting photocatalyst sheet of claim 4, wherein the third metallic photocatalyst comprises silver nanoparticles (NPs) and the fourth metallic photocatalyst comprises cerium NPs.

6. The self-disinfecting photocatalyst sheet of claim 1, wherein the substrate material comprises a glass.

7. The self-disinfecting photocatalyst sheet of claim 1, wherein the substrate material comprises a resin.

8. The self-disinfecting photocatalyst sheet of claim 1, wherein an adhesive layer is coated on the second side of the substrate material.

9. The self-disinfecting photocatalyst sheet of claim 8, wherein the adhesive layer comprises a pressure-sensitive adhesive (PSA) material.

10. The self-disinfecting photocatalyst sheet of claim 8, wherein the adhesive layer comprises an electrostatic-enhancing agent.

11. The self-disinfecting photocatalyst sheet of claim 1, wherein the sheet has a transmission rate of visible light greater than 50%.

12. A self-disinfecting photocatalyst sheet, comprising
a substrate material with a first side and a second side opposite the first side;
a photocatalyst layer comprising a primary photocatalyst and a secondary photocatalyst; and
a layer of a prime material disposed between the substrate material and the photocatalyst layer,
wherein:
the primary photocatalyst comprises a metal oxide photocatalyst,
the secondary photocatalyst comprises a metallic photocatalyst,
a mass ratio of the primary photocatalyst to the secondary photocatalyst is greater than 2:1,
the primary photocatalyst forms a covalent bond with the prime material at a molecular level,
the layer of the prime material is coated on the first side of the substrate material,
the photocatalyst layer is photocatalytic active to every wavelength band of a plurality of wavelength bands comprising 190˜280 nm, 280˜315 nm, 315 400 nm, and 400˜700 nm,
the photocatalyst layer is more photocatalytic active to the wavelength band 190˜280 nm than to the wavelength band 280˜315 nm,
the photocatalyst layer is more photocatalytic active to the wavelength band 280˜315 nm than to the wavelength band 315˜400 nm, and
the photocatalyst layer is more photocatalytic active to the wavelength band 315˜400 nm than to the wavelength band 400˜700 nm.

13. The self-disinfecting photocatalyst sheet of claim 12, wherein the primary photocatalyst further comprises anatase titanium dioxide (TiO₂).

14. The self-disinfecting photocatalyst sheet of claim 12, wherein the secondary photocatalyst further comprises silver, gold, copper, zinc, nickel, cerium, or a combination thereof.

15. The self-disinfecting photocatalyst sheet of claim 14, wherein the secondary photocatalyst further comprises a third metallic photocatalyst and a fourth metallic photocatalyst with no other metallic photocatalysts.

16. The self-disinfecting photocatalyst sheet of claim 15, wherein the third metallic photocatalyst comprises silver nanoparticles (NPs) and the fourth metallic photocatalyst comprises cerium NPs.

17. The self-disinfecting photocatalyst sheet of claim 12, wherein the substrate material comprises a glass.

18. The self-disinfecting photocatalyst sheet of claim 12, wherein the substrate material comprises a resin.

19. The self-disinfecting photocatalyst sheet of claim 12, wherein an adhesive layer is coated on the second side of the substrate material.

20. The self-disinfecting photocatalyst sheet of claim 19, wherein the adhesive layer comprises a pressure-sensitive adhesive (PSA) material.

21. The self-disinfecting photocatalyst sheet of claim 19, wherein the adhesive layer comprises an electrostatic-enhancing agent.

22. The self-disinfecting photocatalyst sheet of claim 12, wherein the sheet has a transmission rate of visible light greater than 50%.
